(19) United States
(12) Patent Application Publication
(10) Pub. No.: US 2015/0011894 A1
(43) Pub. Date: Jan. 8, 2015

(54) SYSTEM FOR AND METHOD OF QUANTIFYING ON-BODY PALPITATION FOR IMPROVED MEDICAL DIAGNOSIS

(71) Applicant: The Regents of the University of California, Oakland, CA (US)

(72) Inventors: Majid Sarrafzadeh, Anaheim Hills, CA (US); Mahsan Rofouei, Los Angeles, CA (US); Mike Sinclair, Kirkland, WA (US)

(21) Appl. No.: 14/367,178

(22) PCT Filed: Dec. 19, 2012
(86) PCT No.: PCT/US12/70708

§ 371 Date: Jun. 19, 2014

Related U.S. Application Data
(60) Provisional application No. 61/577,622, filed on Dec. 19, 2011.

(51) Int. Cl. A61B 5/00; G06T 7/00; A61B 5/11

(52) U.S. Cl. CPC A61B 5/0077; A61B 5/1102; A61B 5/7275; G06T 7/0073; G06T 2210/41; G06T 2207/10004
USPC 600/476

(57) ABSTRACT

A haptic sensor for performing palpation includes a deformable membrane having a reflective surface, a light source, a camera, and a processor. When the sensor is pressed against an object on a body, the deformable membrane deforms to contour to the shape of the object, light is reflected off the reflective surface, and captured by a camera. The reflected light is processed to reconstruct a 3-D image of the object. The rendered image can show abnormalities such as cysts, tumors, or other abnormalities, as well as arterial pressure pulses. In different embodiments, the sensor illuminates the deformed membrane from multiple directions, using shape-from-shading or grayscale mapping, or using video streams to provide more accurate images. The sensor is able to be included as part of a mobile device, such as a mobile phone, thereby making it compact and portable.

### CROSS REFERENCE TO RELATED APPLICATIONS

This application claims priority under 35 U.S.C. §119(e) of the co-pending U.S. provisional patent application Ser. No. 61/577,622, filed Dec. 19, 2011, and titled “System for and Method of Quantifying On-Body Palpitation for Improved Medical Diagnosis,” which is hereby incorporated by reference.

### FIELD OF THE INVENTION

This invention relates to object imaging. More particularly, this invention relates to reconstructing three-dimensional images from palpitations for medical purposes.

### BACKGROUND OF THE INVENTION

Palpation is a traditional diagnostic procedure in which physicians use their fingers to externally touch and feel body tissues. Palpation is used as part of a physical examination to determine the spatial coordinates of an anatomical landmark, assess tenderness through tissue deformation, and determine the size, shape, firmness and location of an abnormality in the body through the tactile sensing of elasticity modulus differences. Palpation can be used in finding tumors, arteries, moles, or other objects on the body.

Unfortunately, palpation is subjective inasmuch as the results may vary among physicians. The results depend on the physician's ability and experience, all of which make the results prone to error.

One system, described in U.S. Pat. No. 5,459,329 to Sinclair, uses a single light source, a deformable membrane having a reflective surface, and a camera. The object is pressed against the membrane to deform the membrane, the light source illuminates the reflective surface, and the light reflected from the surface is captured by the camera, processed, and used to identify the contours of the object. Sinclair does not disclose any algorithms for accurately rendering microscopic structures and arterial pressure pulses, nor is it capable of being packaged in a mobile, low-cost package.

### SUMMARY OF THE INVENTION

In a first aspect of the invention, a system for reconstructing a three-dimensional image includes a deformable membrane that contours to a shape of at least a portion of an object, the deformable membrane having a reflective surface; a camera positioned to receive illumination reflected from the reflective surface; a light source for illuminating the reflective surface from multiple directions relative to a fixed position of the camera; and a processor for reconstructing a three-dimensional image of the shape from illumination reflected from the reflective surface.

In one embodiment, the system also includes a controller. The controller sequentially illuminates the reflective surface from the multiple directions and also causes the camera to sequentially take images of the shape from the illumination reflected from the reflective surface. In another embodiment, the light source includes a plurality of light-emitting diodes equally spaced from each other.

In a second aspect, a system for reconstructing a three-dimensional image includes a deformable membrane that contours to a shape of at least a portion of an object, the deformable membrane having a reflective surface; a camera positioned to receive illumination reflected from the reflective surface; a single-light source for illuminating the reflective surface; and a processor for reconstructing a three-dimensional image of the shape from illumination reflected from the reflective surface using a shape-from-shading algorithm. The shape-from-shading algorithm includes a brightness constraint, a smoothness constraint, an intensity gradient constraint, or any combination thereof.

In a third aspect, a system for reconstructing a three-dimensional image includes a deformable membrane that contours to a shape of at least a portion of an object, the deformable membrane having a reflective surface; a camera positioned to receive illumination reflected from the reflective surface; a single-light source for illuminating the reflective surface; and a processor for reconstructing a three-dimensional image of the shape from illumination reflected from the reflective surface using grayscale mapping.

In a fourth aspect, a system for reconstructing a three-dimensional image includes a deformable membrane that contours to a shape of at least a portion of an object, the deformable membrane having a reflective surface; a camera positioned to receive illumination reflected from the reflective surface; a light source for illuminating the reflective surface to produce reflected light onto the camera; and a processor for reconstructing a three-dimensional image of the shape from a video stream corresponding to illumination reflected from the reflective surface.

In a fifth aspect, a system for making medical diagnoses includes a computer-readable medium containing computer-executable instructions that when executed by a processor perform the method correlating one or more three-dimensional images of a body location with a stored medical diagnosis. In one embodiment, the system also comprises a library that maps differences between three-dimensional images of a body location to medical diagnoses.

### BRIEF DESCRIPTION OF THE DRAWINGS

FIGS. 1A-C show different views of a haptic sensor in accordance with one embodiment of the invention.

FIG. 2 shows a top-cross-sectional view of the haptic sensor in FIGS. 1A-C.

FIGS. 3A-C are photographs showing the results of light direction calibration on a sphere input in accordance with one embodiment of the invention.

FIG. 4 is a photograph showing the results of light direction calibration on a 4-sphere plate input in accordance with one embodiment of the invention.

FIG. 5 is a graph showing the results of a photometric stereo surface reconstruction on a 4-sphere plate pressed against the membrane of the haptic sensor of FIGS. 1A-C.

FIG. 6 is a top cross-sectional view of a haptic sensor in accordance with one embodiment of the invention.

FIG. 7 is an exploded view of a haptic sensor in accordance with one embodiment of the invention.

FIG. 8 shows a haptic sensor implemented on a mobile phone in accordance with one embodiment of the invention.

FIGS. 9A-E show graphs of different pulse shapes for determining arterial pulse characteristics in accordance with one embodiment of the invention.

FIG. 10 shows the steps of an algorithm for arterial pulse palpation in accordance with one embodiment of the invention.

FIGS. 11A and 11B are photographs of a sample frame indicating the location of an arterial pulse and a 3-D image of the arterial pulse, respectively, generated in accordance with one embodiment of the invention.

FIG. 12 is a graph of a heart rate used to illustrate one embodiment of the invention.

FIG. 13 is a graph showing a signal obtained by projecting frames onto a primary basis image in accordance with one embodiment of the invention.

FIG. 14 is a graph illustrating fitting individual heartbeat segments into a two-peak Gaussian Mixed Model.

FIGS. 15A-C are graphs of user results for arterial pulse characteristics for 3 different users obtained using the pulse algorithms in accordance with one embodiment of the invention.

### DETAILED DESCRIPTION OF THE INVENTION

A haptic sensor in accordance with embodiments of the invention is a low-cost device that enables the real-time visualization of the haptic sense of elastic modulus boundaries, which is essentially the tissue deformation caused by a specific force. The sensor captures images that describe the three-dimensional (3-D) position and movement of underlying tissue during the application of a known force, essentially what a physician feels through manual palpation.

The sensor and supporting software enable the visualization and documentation of the equivalent of 3-D tactile input from a known applied force. The sensor eliminates the subjective analysis of physical palpation examinations and gives more accurate and repeatable results, yet is less expensive to implement than MRI, ultrasound, or similar techniques.

Data processed from captured images is also a good means for documentation for patient records. This way, physicians are also able to objectively measure change over time by comparing past data. By incorporating image registration techniques, it is possible to accurately assess change over time. Physicians can also share extracted features of abnormalities together with captured images and data with other physicians for further research.

The haptic device is also able to be used to teach medical palpatory diagnosis. One such implementation is the Virtual Haptic Back (VHB), a virtual reality tool for teaching clinical palpatory diagnosis of the human back. Using embodiments of the invention, less experienced physicians or medical students are able to enhance their palpation perception by comparing their assessments with accurate quantitative assessments from the sensor.

The system can also be used for virtual palpation in tele-medicine and to develop applications such as remote diagnosis of medical conditions for use in rural locations.

The systems described herein have applications in palpating different body parts and improving diagnosis. The systems have applications including, but not limited to, the following areas:
Palpating masses such cysts and abnormalities for cancer detection
Assessing body tissue stiffness
Pulse palpation for use in Chinese medicine
Arterial pressure pulse palpation
Pulse wave velocity measurements used for prediction of cardiovascular diseases such as arterial stiffness
Palpating and assessment of the thyroid
Teaching palpatory skills to medical students
Tele-medicine, used in rural and other remote locations
Documentation and recording of medical assessments

### Photometric Stereo Sensor

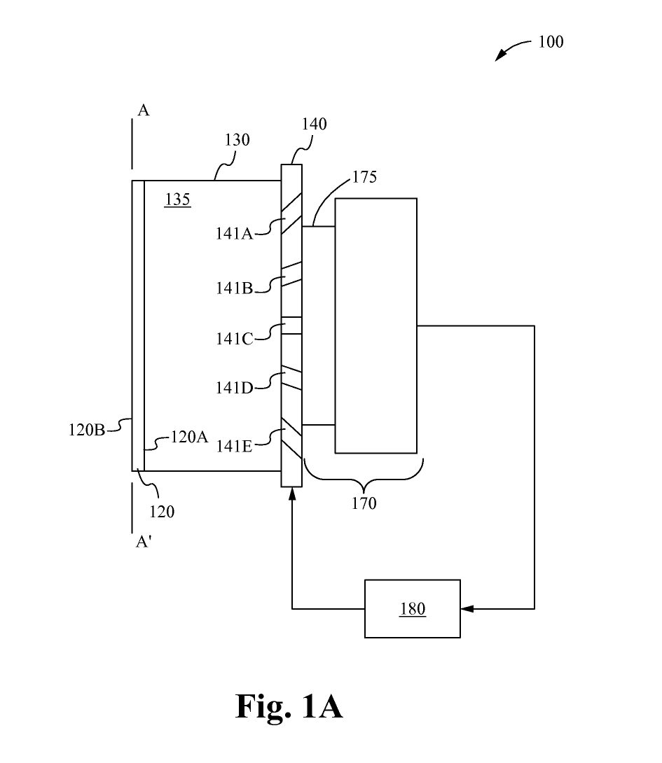

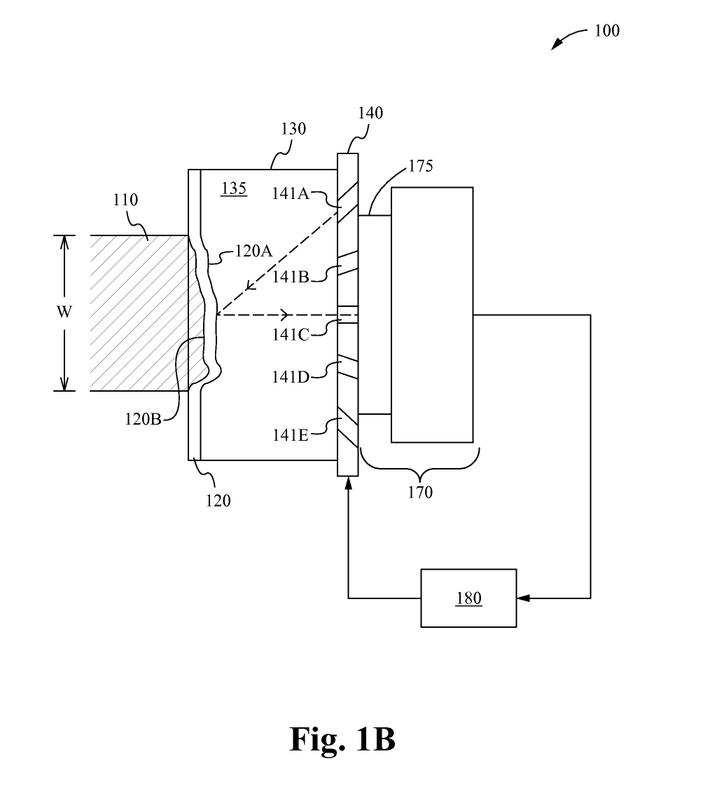

FIG. 1A is a side cross-sectional view of a haptic sensor 100 in accordance with one embodiment of the invention. The haptic sensor 100 uses a photometric stereo approach to 3-D image reconstruction. It uses multiple images taken from the same viewpoint but under different illumination directions to estimate local surface orientation. The change in intensities in the images depends on both local surface orientation and illumination direction. The sensor 100 includes a flexible, deformable membrane 120 having a reflective surface 120A and a surface 120B opposite the reflective surface; a hollow cylinder 150 having a cavity 135; an annulus (“light ring”) 140 housing 8 equi-spaced light-emitting diodes (LEDs) 141A-H, some of which are eclipsed in the figure; a camera 170 having a lens 175; and a controller and image processor 180. The flexible membrane 120 covers or is adjacent to a first end of the cylinder 130, and the light ring 140 is coupled to a second end of the cylinder 130. The light ring 140 couples the second end of the cylinder 130 to the lens 175. The reflective surface 120A faces into the cavity 135 and thus faces the LEDs 141A-H. The LEDs 141A-H are pointed or otherwise arranged to illuminate the cavity 135 at angles to the normal of the first end of the cylinder 130, to thereby illuminate the reflective surface 120A from multiple directions. The output of the camera 170 is coupled to the controller and image processor 180, which is operatively coupled to the LEDs 141A-H.

FIG. 1B shows the sensor 100 when an object 110 is inserted through the first end of the cylinder 130, pressing against the surface 120B to thereby deform the membrane 120. In all the figures, the same reference label refers to the same or identical element. As shown in FIG. 1B, the flexible membrane 120 deforms or contours to the shape of that portion of the object 110 pressing against it. FIG. 1B shows light from the LED 141A illuminating the reflective surface 120A from one direction and reflected off the reflective surface 120A to the lens 175.

In one embodiment, the controller and image processor 180 performs multiple functions: It sequentially turns the LEDs 141A-H ON and OFF such that only one of the LEDs 141A-H is ON at a time. It uses the digital pixels captured by the camera 170 to reconstruct a 3-D image of that portion of the object 110 pressing against the flexible membrane 120. While the LEDs 141A-H are sequentially turned ON and OFF, the lens 175 is held stationary relative to the reflective surface 120A. FIG. 1C is an exploded view of a portion of the sensor 100.

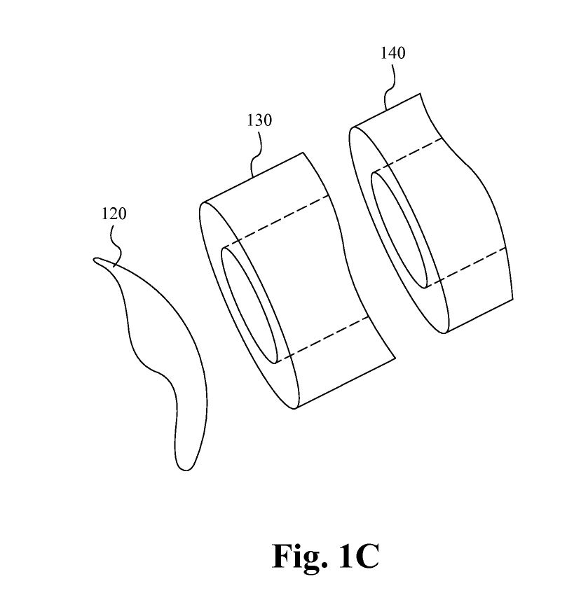

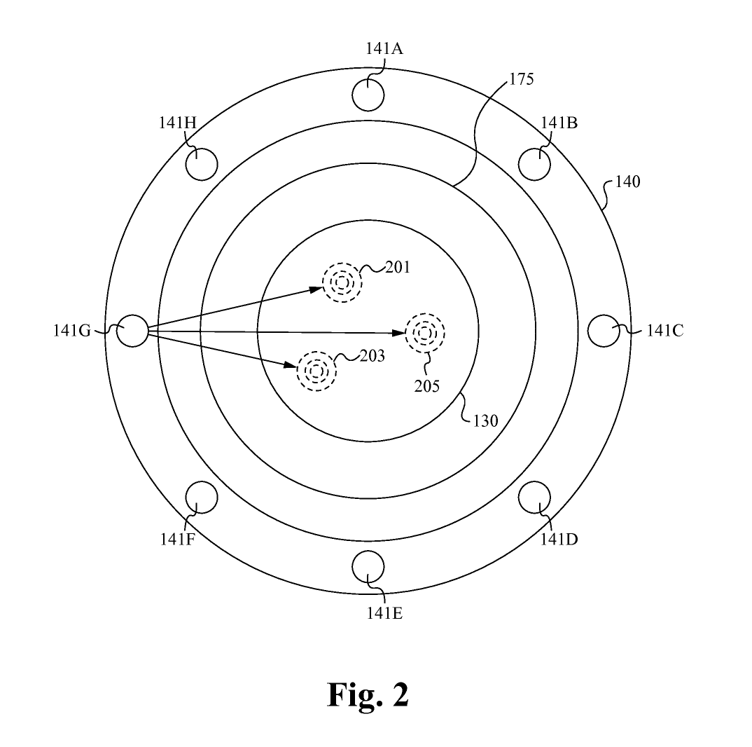

FIG. 2 is a top perspective view of the system 100 taken along the line AA′, shown in FIG. 1A. The lens 175, the light ring 140, and the cylinder 130, are concentric. The inside diameter of the light ring 140 is larger than the outside diameter of the cylinders 130, thereby allowing the light ring 140 to aim light, at different angles, into the cavity 135. FIG. 2 shows illumination from the LED 141G reflected off contours 201, 203, and 205 of the object 110 and on to the lens 175. In the embodiment of FIG. 2, to ensure that a sufficient amount of reflected light reaches the lens 175, the inner diameter of the light ring 140 is larger than the width W of the object 110.

In photometric stereo, multiple images are taken while holding the viewing direction constant. Since there is no change in imaging geometry, all picture elements (x,y) correspond to the same point in all images. The effect of changing light direction is to change the reflectance map. Therefore with multiple equations (a minimum of three), the following equations can be solved:

I1(x,y)=R1(p,q) Equation (1)

I2(x,y)=R2(p,q) Equation (2)

I3(x,y)=R3(p,q) Equation (3)

It will be appreciated that the processor 180 solves Equations 1-3 to reconstruct the 3-D image of that portion of the object 110 pressing against the membrane 120.

In diffuse reflections, Equations 1-3 can be written as I=KdN.L where Kd is the albedo. With more light sources, the reconstruction results are more accurate.

When implementing this method, two calibrations for a standard photometric stereo algorithm are performed. First, the camera 170 must be calibrated to obtain the scene irradiance from measured pixel values. Second, lighting directions and intensities must be known to uniquely determine the surface. With these two calibrations, surface orientations and albedos can be estimated uniquely from three images for Lambertian scenes.

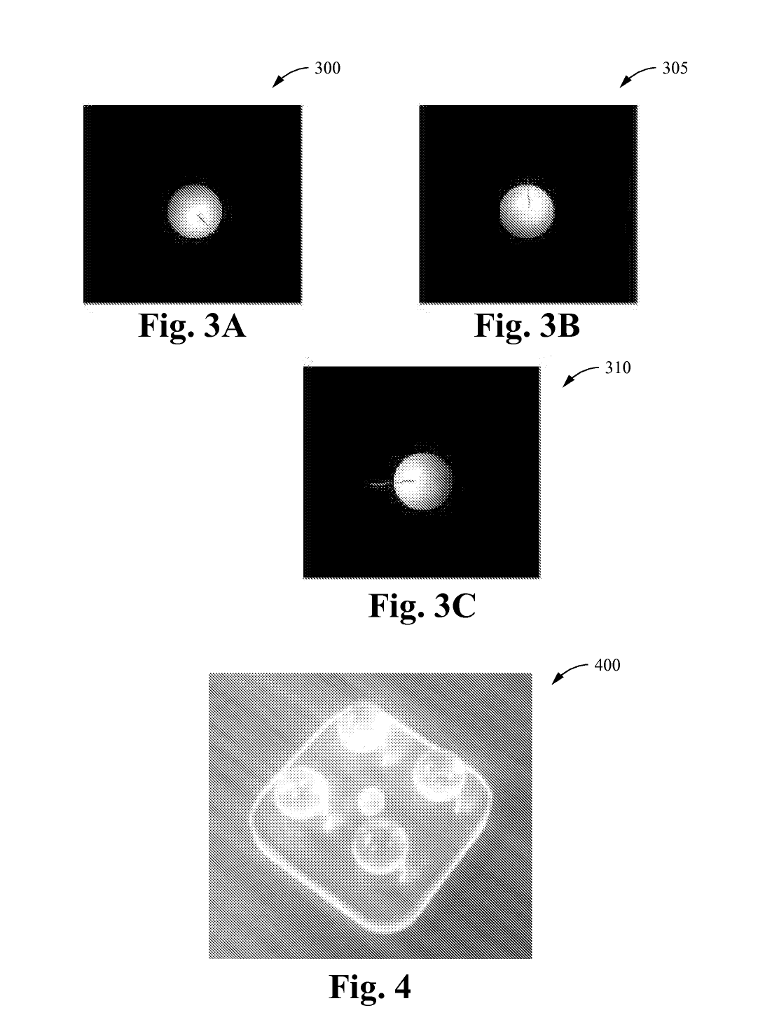

FIGS. 3A-C are photographs 300, 305, and 310, respectively, showing the results of light direction calibration performed on three input images using the sensor 100.

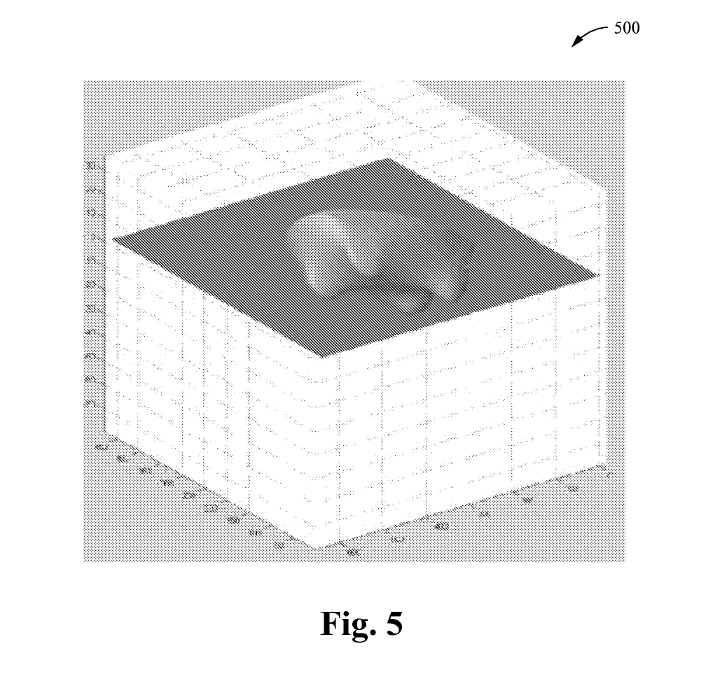

In one experiment, the light direction calibration for the sensor 100 was performed for all the 8 light views, and the results were used to reconstruct a 3-D image of a slide 400, shown in FIG. 4. The slide 400 has 4 equal spheres, representing cysts, placed within equal distances of each other. FIG. 5 shows the photometric stereo 3-D reconstruction 500 of the slide 400 using the haptic sensor 100.

It will be appreciated that FIGS. 1A, 1B, and 2 are merely illustrative of one embodiment used to illustrate the principles of the invention. Those skilled in the art will recognize many variations. For example, while the light ring 140 houses 8 LEDs, any number of LEDs can be used, preferably at least 3, with more LEDs producing more accurate reconstructed images. Volumes other than hollow cylinders (e.g., 130) can be used to support the flexible membrane 120 or to house the LEDs 141A-H or other components. Separate modules can be used to control the LEDs 141A-H, to map the reflected light to digital data suitable for image processing, and to construct 3-D images from the digital data. Light sources other than LEDs (e.g., 141A-H) can be used to illuminate the deformed reflective surface 120A. An inner diameter of the light ring 140 does not have to be larger than an outer diameter of the cavity 135. In alternative embodiments, lenses are used to focus light onto the reflective surface of the flexible membrane.

### Photometric Shade-from-Shading Sensor

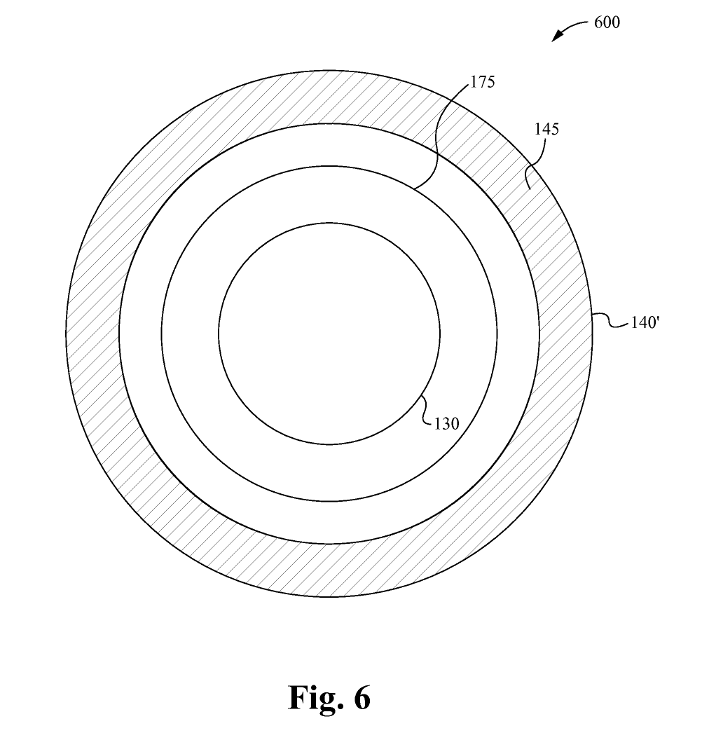

In another aspect of the invention, the multiple light sources of FIG. 1A (e.g., LEDs 141A-H) are replaced with a single-light source, and 3-D images are reconstructed by a processor (e.g., 180) using a shape-from-shading algorithm. FIG. 6 shows a top cross-sectional view of a sensor 600 in accordance with one embodiment of the invention. The sensor 600 is similar to the sensor 100, except the light ring 140′ includes a single, circular light LED 145, and the corresponding controller and image processor (not shown) perform different algorithms, discussed below.

Shading plays an important role in human perception of shape. Shape from shading aims to recover shape from gradual variations of shading in one two-dimensional (2-D) image. This is generally a difficult problem to solve because it corresponds to a linear equation with three unknowns. In accordance with one embodiment, a unique solution to the linear equation is found by imposing certain constraints.

In solving shape from shading and representing 3-D data using gradients, since each surface point has two unknowns for the surface gradient, and each pixel provides only one gray value, the system is “under determined.” To overcome this limitation, embodiments of the invention impose any one or more of a brightness constraint, a smoothness constraint, and an intensity gradient constraint. These constraints make this reconstruction method less accurate, but much easier to construct, than the photometric stereo approach discussed in relation to FIGS. 1A-C.

In operation, when an object is pressed against the flexible membrane 120 to deform the reflective surface 120A, the controller and image processor 180 controls the LED 145 to illuminate the reflective surface 120A. From the illumination reflected from the flexible membrane 120, the camera 170 captures a single 2-D image of the deformed flexible membrane 120. Using one or more of a brightness constraint, a smoothness constraint, and an intensity gradient constraint, the controller and image processor processes the captured 2-D image to reconstruct a 3-D image of the portion of the object pressing against the flexible membrane 120.

It will be appreciated that this example is used merely to illustrate the principles of the invention. After reading this disclosure, those skilled in the art will appreciate that changes can be made to the example in accordance with the principles of the invention. For example, constraints other than brightness, smoothness, and intensity gradient can be imposed to overcome the under determinedness of the 3-D image reconstruction algorithm.

### Photometric Grey-Scale Sensor

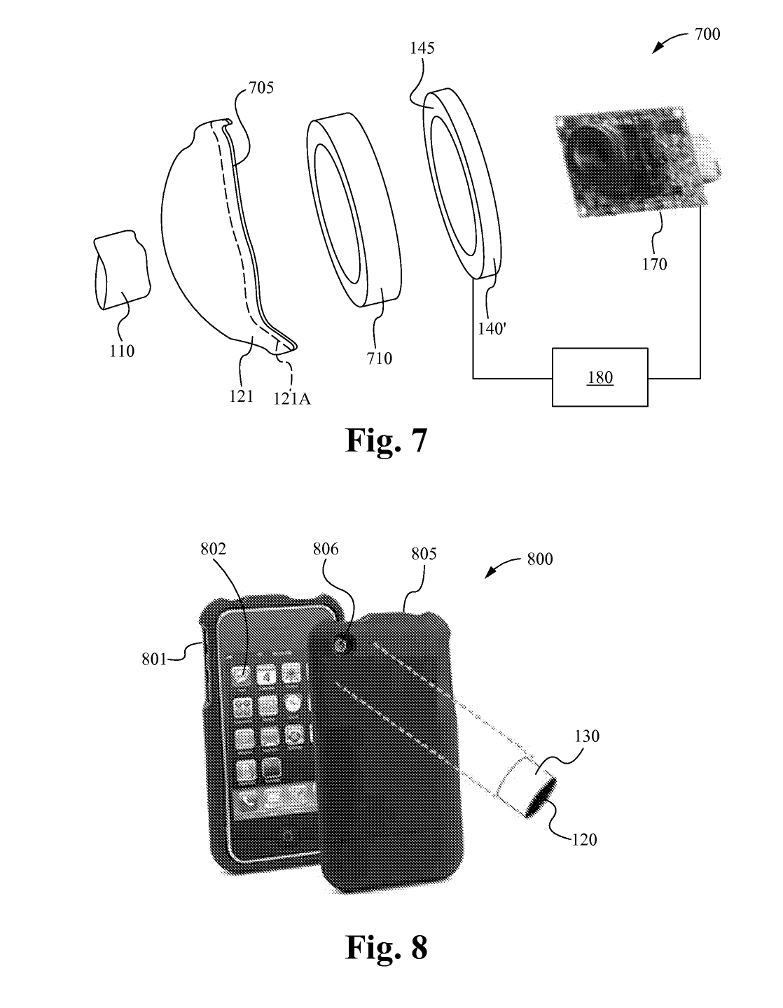

In another aspect of the invention, an elastomer is measured for strain to determine the 3-D image of an object pressed against it. FIG. 7 is an exploded view of a haptic sensor 700 in accordance with one embodiment of the invention. The sensor 700 includes a white deformable membrane 121, an isotropically dyed elastomer 705 attached to an inner surface 121A of the membrane 121, a clear, rigid face plate 710, a light ring 140′ housing a single-light source 145, a camera 170, and a processor 180. In some embodiments, the white deformable membrane 121 is itself reflective and thus forms the reflective surface 121A. The elastomer 705 is able to be measured for strain, so that it is known how it changes shape relative to force. This embodiment is then able to be used in stiffness and tenderness assessment.

In operation, an object of interest 110 is pressed externally against the membrane 121, which is illuminated as described above. This results in the 3-D deformation of the membrane 121 and the attached dyed elastomer 705 and finally in the grayscale image representing the 3-D depth map of the object 110 captured by the camera 170. Different parts of the object 110 are deformed at different depths from the face plate 710, proportional to the local applied force and inversely by the modulus of the object 110. This 3-D deformation of the optically attenuating elastomer 705 causes the illumination to pass through varying thicknesses and hence varying attenuations as seen by the camera 170. The smaller the distance the light has to travel through the elastomer 705, the lighter it appears. Therefore positions on the reflecting white membrane 121 which are deformed to be nearer to the face plate 710 appear lighter than positions farther away. This results in a function that maps membrane deformation heights at each pixel location to the grey-scale intensity value of the camera 170 at that location. The sensor 700 thus functions as a real-time 3-D surface digitizer.

The haptic sensor 700 is merely illustrative of one embodiment of the invention. In another embodiment, the dyed elastomer 705 is replaced by a liquid contained within the deformable membrane 121. In one embodiment, the light ring 145 is replaced by a different illumination source (e.g., source 141A-H) configured to produce sufficient light to impinge on (1) the isotropically dyed elastomer 705, (2) a liquid, or (3) a functionally similar element, to reflect off the membrane surface 121A, and back to the camera 170. After reading this disclosure, those skilled in the art will recognize other variations that can be made in accordance with the principles of the invention.

### Mobile Implementations

The embodiments of the invention are able to be implemented on a mobile device, such as a suitably configured mobile phone, thus allowing diagnosticians to carry a haptic sensor with them wherever they go. FIG. 8 shows a mobile haptic sensor 800 that includes a mobile phone 801 and accompanying case 805 in accordance with one embodiment of the invention. The sensor 800 uses the built-in camera of the phone 801 as an image sensor (e.g., 170, FIG. 1A). In some configurations (e.g., sensors 100 and 700), the flashlight on the mobile phone 805 is the illumination source (e.g., 141A-H or 145). The case 805 includes an aperture 806 that houses the hollow cylinder 130, covered by the deformable membrane 120. A processor on the mobile phone (not shown) functions as the processor (e.g., 180) for image processing and 3-D reconstruction, as described above. The reconstructed 3-D image is able to be viewed on a display 802 of the phone 801.

### Arterial Pressure Pulse Extraction

In accordance with another aspect of the invention, a haptic sensor is able to generate 3-D images of arterial pulse pressure waveforms. Any of the haptic sensors discussed above are able to be used in accordance with this aspect, with the image reconstruction algorithm discussed below. As one example, a haptic sensor in accordance with this aspect includes a white deformable membrane, an isotropically dyed elastomer or liquid, a clear rigid faceplate, an illumination source, a camera, and a processor, such as the sensor 700. Unlike prior-art pressure sensor based methods, embodiments of the invention increase accuracy to the pixel level and are portable, non-invasive, and low-cost.

Arterial pulse pressure is considered a fundamental indicator for diagnosis of several cardiovascular diseases. An arterial pulse waveform can be acquired by palpation on different areas on the body such as a finger, a wrist, a foot, or a neck. Pulse palpation is also considered a diagnostic procedure used in Chinese medicine.

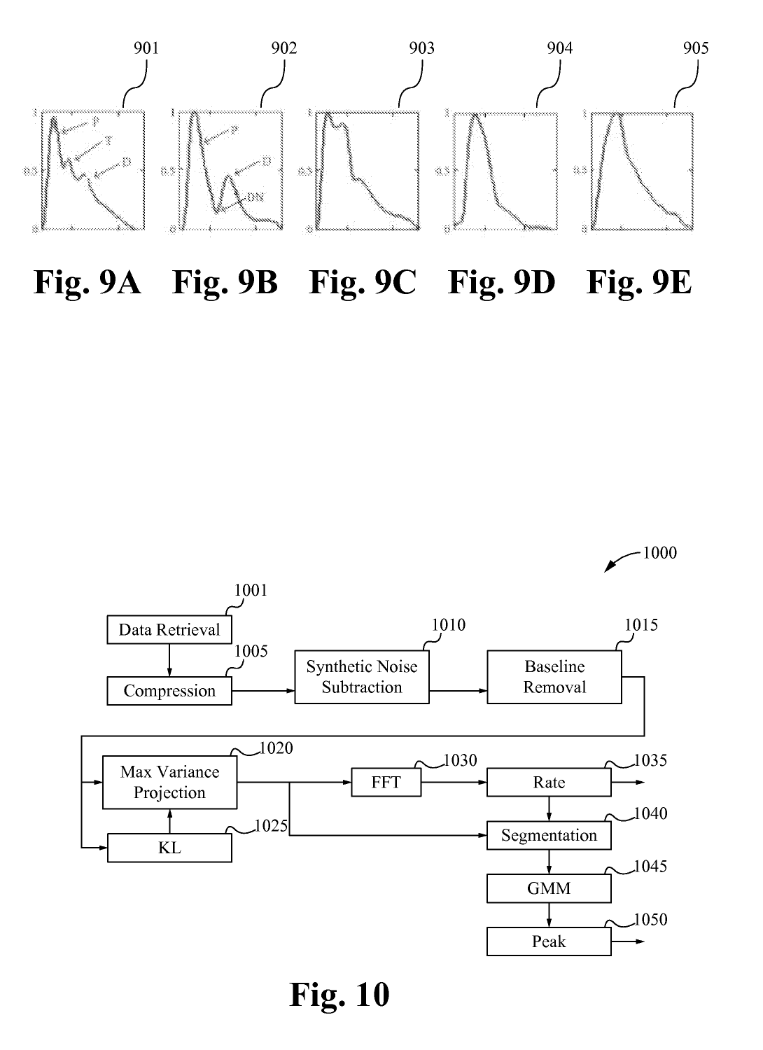

The waveform acquired by palpation is considered to offer more information than the single pulse waveform from an electrocardiogram (ECG). The ECG signal only reflects bio-electrical information of the body while a pulse palpation signal, especially at different locations along an artery, reveals diagnostic information not visible in ECG signals. Different kinds of pulse patterns are defined based on different criteria such as position, rhythm, shape, etc. From shape perspectives, all of the pulses can be defined according to the presence or absence of three types of waves, a P (Percussive or primary) wave, a T (tidal or secondary) wave, and a D (Dicrotic or triplex) wave. FIGS. 9A-E show examples of segment pulses 901-905, respectively, with the presence or absence of these waves.

The percussion, tidal, and dicrotic waves can be indicators of specific conditions. For example, they can indicate the decrease in compliance of small arteries and the elasticity of blood vessel walls. In addition to the shape of the pressure pulse features such as width and rate, the position of the pulse is also important. Measuring pulse propagation time along the artery is also important in measuring blood velocity.

FIG. 10 shows the steps 1000 of an algorithm to extract arterial pressure pulse from the output image of a membrane pressed against the palpatory area of the pulse on the hand in accordance with one embodiment of the invention. This embodiment uses video streams rather than single images to detect temporal changes.

Referring to FIG. 10, in the step 1001, data are collected from the image sensor. In one embodiment, data are collected at a rate of 60 frames per second (fps), with a frame resolution of 640×480 pixels. To lower the computational complexity of the algorithm, in the step 1005, the data are compressed by down-sampling the frame size to 128×96 pixels. In the step 1010, an initial empty frame is used to subtract unwanted artifacts from the images.

Next, in the step 1015, a baseline removal step is performed. Baseline drift is visible in raw data. This is due to applied pressure variations from human movement. Multiple schemes are available for advanced baseline removal procedures, however it was experimentally determined that simple time-domain high-pass filtering with a cut-off frequency of 0.5 Hz can perform reasonably well under slow movement conditions. This filtering will not remove sudden movements which are in the passband. Slower baseline variations such as those induced as a result of breathing movements are removed by the filter.

The baseline removal step 1015 is followed by parallel maximum variance projection 920 and Karhunen-Loeve (KL) transform 1025 steps. The output of the maximum variance projection step 920 is input to a Fast-Fourier Transform (FFT) 930 and a segmentation step 940. The output of the FFT 930 is input to a rate analysis step 935, which generates an output for a segmentation step 940. The output of the segmentation step 940 is input to a Gaussian Mixed Model (GMM) step 945, whose output is used to generate peak statistics 950, used to generate a 3-D image.

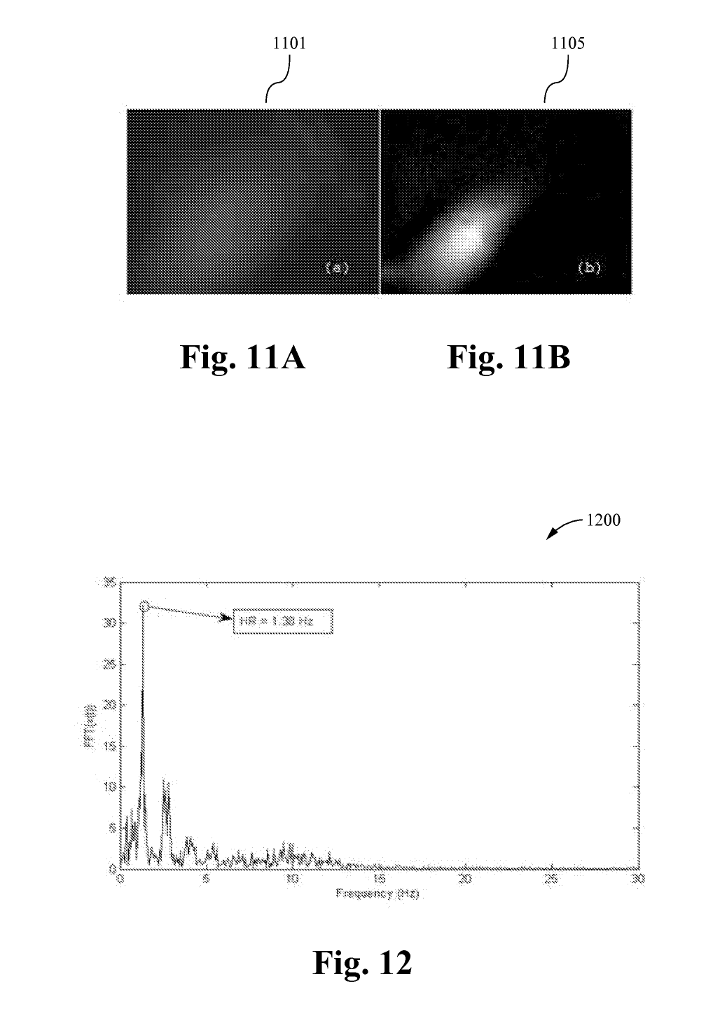

Equations 4-6 illustrate the mathematics behind one embodiment of the invention, and FIGS. 11A-B, 12-15, and 15A-E associated results in determining heart rate. The following explanation discusses some of the steps 1000 in more detail.

In one embodiment, compressed image data at frame n is expressed by Vₜ_{c}(m,n), where V^{t}_{c}(m,n) represents a 128×96 matrix of grayscale pixel data. The output of the baseline removal block is then represented as a convolution:

$${X}_{BC}^{t} ( m , n ) = {\sum }_{τ = 0}^{t} {X}_{c}^{τ} ( m , n ) \cdot {h}_{HP} ( t - τ ) \qquad Equation ( 4 )$$

where h_{HP}(t) is the impulse response of the high-pass filter. Next, a one-dimensional (1-D) function of time x(t) is extracted from the 3-D X^{t}_{BC}(m,n) image data.

In the embodiment of FIG. 10, the Karhunen-Loeve (KL) transform is used to obtain x(t) as shown in Equation (5):

x(t)=w₁^{T}\tilde{x}_{BC}(t) Equation (5)

where

\tilde{x}_{BC}(t)

is a vector obtained by columnization of the matrix X^{t}_{BC}(m,n), and w₁ is the first eigenvector of the covariance matrix

$${C}_{\tilde{x}} = E ⌊ {\tilde{x}}_{BC} , {\tilde{x}}_{BC}^{T} ⌋$$

corresponding to the largest eigenvalue. Implied in this scheme is the modeling of video data as a stochastic process in time, where projecting the frames onto the first orthogonal basis image w₁ obtained by the KL transform maximizes the variance of the output process x(t). Also implied is the treatment of variance as a measure of information.

While simpler approaches such as a simple summation over the image may be feasible in many instances, there are cases where these approaches will not provide sufficient precision. This occurs, for example, where an increased pressure on the membrane of the apparatus causes the liquid in the membrane to shift from one location to another, resulting in a near-zero net pixel brightness effect on the entire frame image. The KL approach in such cases ensures that the relevant data are captured appropriately by assigning negative weights to some of the pixels. The KL transform also emphasizes the image locations where most of the variation is happening, mostly discarding areas unaffected by the heartbeat pulses. FIG. 11A shows a haptic lens sample frame compared to the primary basis image obtained by a KL transform according to FIG. 10, used for data extraction shown in FIG. 11B. The KL transform provides most accurate results with the baseline removal step 1015.

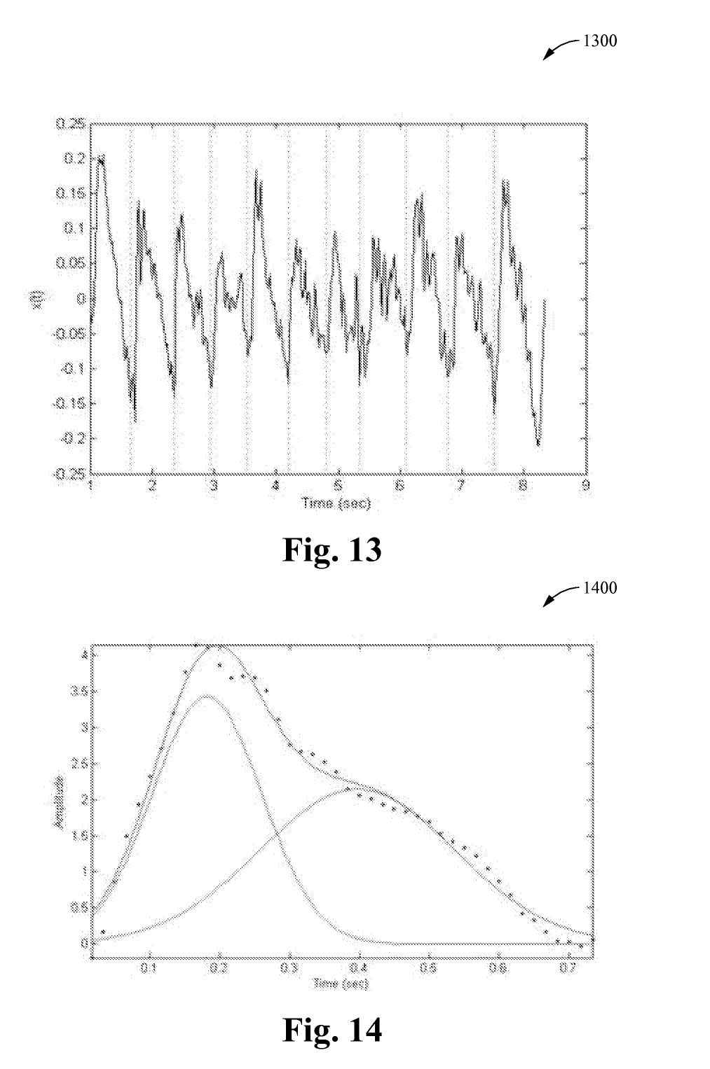

In this example, the heart rate is derived by first performing the FFT (step 1030) followed by a peak search in the interval 0.7 to 2 Hz, as shown by the graph 1200 in FIG. 12. Using the heart rate, the x(t) signal is then divided into segments (step 1040) representing heartbeats, as shown by the graph 1300 in FIG. 13. In FIG. 13, the detected segments are separated by vertical dotted lines.

The segment separation is performed by finding consecutive minimums separated by heartbeat period intervals (as derived from the FFT step 930), with a tolerance of 10%. The segments are next averaged and fitted to a set of Gaussian Mixed Models (step 1045) with multiple peaks, with each set representing one of the pulse models shown in FIGS. 8A-E:

$$\overline{x} ( t ) = \text{?} {α}_{k} \cdot \text{?} + {v}_{m} ( t ) \\ \text{?} \text{indicates text missing or illegible when filed} \qquad Equation ( 6 )$$

where

x̅(t)

represents one heartbeat segment obtained by averaging over the individual segments obtained from x(t), Nₘ is the number of peaks in the mth pulse model, αₖ, μₖ, and σₖ are the optimization variables in the fitting process (step 945), and vₘ(t) is the error signal. In one embodiment, the fitting procedure is performed using the Nelder-Mead iterative method as shown by the graph 1400 in FIG. 14, which shows fitting individual heartbeat segments into a two-peak GMM model. The mean square error obtained from each fitting result is computed and compared to decide the pulse model (e.g., FIGS. 8A-E).

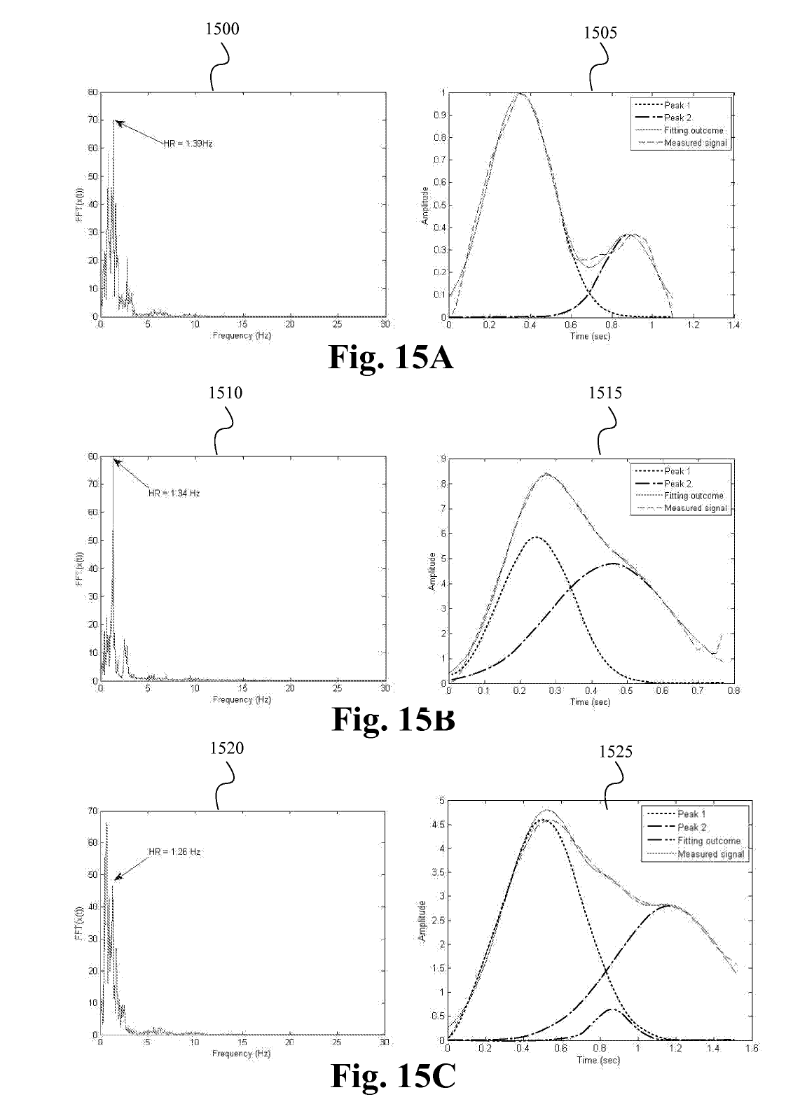

FIGS. 15A-C show test results for frequency domain analysis and curve-fitting procedures for 3 different users, generated using the algorithm 1000. Users were asked to adjust the location of the sensor on their wrists until they could see pulse synchronization in the image shown on a computer screen. FIG. 15A shows a graph 1500 of a user pulse rate and a corresponding graph 1505 of a pulse rate measured using the algorithm 1000, at 1.39 Hz. FIG. 15B shows a graph 1510 of a user pulse rate and a corresponding graph 1510 of a pulse rate measured using the algorithm 1000, at 1.34 Hz. FIG. 15C shows a graph 1520 of a user pulse rate and a corresponding graph 1525 of a pulse rate measured using the algorithm 1000, at 1.26 Hz.

It will be appreciated that the examples are merely illustrative. For example, the steps 1000 can be performed in different orders, some steps can be added, and other steps can be deleted. The peak search can be in an interval different from 0.7 to 2 Hz. The data collection rate can be more than or less than 60 fps. The downsampling can be to a different frame size.

In other embodiments, 3-D images are stored in a library and correlated with diagnoses.

As one example, a system takes a one 3-D image and makes a diagnosis corresponding to characteristics of the image, such as its location, size, and shape. A growth in the throat having a certain size and shape can correspond to a malignant tumor. In another embodiment, a 3-D images of an object (e.g., a growth) at a particular body location is compared to a library of previously captured 3-D images of objects at the same location. The system correlates differences between the images to make diagnoses. A patient's health can thus be tracked over time, such as by determining that a growth is growing larger, growing smaller, or spreading. Preferably, the system has a memory containing computer-executable instructions for performing the algorithms associated with these embodiments and a processor for executing these instructions.

In operation, a haptic sensor in accordance with embodiments of the invention is pressed against a portion of a patient's body. A 3-D image of the object is rendered, allowing physicians to make accurate, objective assessments of, among other things, tissue size, shape, and location.

While the examples shown above are directed to medical diagnoses, it will be appreciated that the invention is not limited in this way. Embodiments of the invention can be used in other fields.

It will be readily apparent to one skilled in the art that other modifications may be made to the embodiments without departing from the spirit and scope of the invention as defined by the appended claims.

We claim:

1. A system for reconstructing a three-dimensional image comprising:
a deformable membrane (120) that contours to a shape of at least a portion of an object (110), the deformable membrane (120) having a reflective surface (120A);
a camera (170) positioned to receive illumination reflected from the reflective surface (120A);
a light source (141A-H) for illuminating the reflective surface (120A) from multiple directions relative to a fixed position of the camera (170); and
a processor (180) for reconstructing a three-dimensional image of the shape from illumination reflected from the reflective surface (120A).

2. The system of claim 1, further comprising a controller (180) for sequentially illuminating the reflective surface (120A) from the multiple directions.

3. The system of claim 2, wherein the controller (180) causes the camera to sequentially take images of the shape from the illumination reflected from the reflective surface (120A).

4. The system of claim 1, wherein the light source (141A-H) comprises a plurality of light-emitting diodes (141A-H) equally spaced from each other.

5. The system of claim 1, wherein reconstructing the three-dimensional image comprises using multiple reflectance maps.

6. The system of claim 1, further comprising a case (805) for a portable electronic device (801), wherein the camera (170), the light source (141A-H), and the processor (180) form part of the electronic device (801), the light source (141A-H) forming a flash for the camera (170), the case (805) having an aperture (806) that houses the deformable membrane (120) and aligns the deformable membrane (120) with the light source (141A-H).

7. The system of claim 6, wherein the portable electronic device (801) comprises a mobile telephone.

8. A method of reconstructing a three-dimensional image comprising:
illuminating a reflective surface (120A) of a deformed membrane (120) from multiple locations relative to a fixed position, wherein the reflective surface (120A) is contoured to a shape of at least a portion of an object (110); and
reconstructing a three-dimensional image of the shape from illumination reflected from the reflective surface (120A).

9. The method of claim 8, wherein illuminating a reflective surface (120A) comprises sequentially illuminating the reflective surface (120A) from the multiple locations.

10. A system for reconstructing a three-dimensional image comprising:
a deformable membrane (120) that contours to a shape of at least a portion of an object (110), the deformable membrane (120) having a reflective surface (120A);
a camera (170) positioned to receive illumination reflected from the reflective surface (120A);
a single-light source (145) for illuminating the reflective surface (120A); and
a processor (180) for reconstructing a three-dimensional image of the shape from illumination reflected from the reflective surface (120A) using a shape-from-shading algorithm.

11. The system of claim 10, wherein the shape-from-shading algorithm includes a brightness constraint, a smoothness constraint, an intensity gradient constraint, or any combination thereof.

12. A method of reconstructing a three-dimensional image comprising:
illuminating a reflective surface (120A) of a deformed membrane (120) using a single-light source (145), wherein the reflective surface (120A) is contoured to a shape of at least a portion of an object; and
reconstructing a three-dimensional image of the shape from illumination reflected from the reflective surface (120A) using a shape-from-shading algorithm.

13. A system for reconstructing a three-dimensional image comprising:
a deformable membrane (121) that contours to a shape of at least a portion of an object (110), the deformable membrane (121) having a reflective surface (121A);
a camera (170) positioned to receive illumination reflected from the reflective surface (121A);
a single-light source (145) for illuminating the reflective surface (121A); and
a processor (180) for reconstructing a three-dimensional image of the shape from illumination reflected from the reflective surface (121A) using grayscale mapping.

14. The system of claim 13, wherein the deformable membrane (121) encloses a flexible material (705).

15. The system of claim 13, wherein the flexible material (705) comprises an isotropically dyed elastomer.

16. The system of claim 15, wherein the flexible material (705) comprises a liquid.

17. The system of claim 13, further comprising a case (805) for a portable electronic device (801), wherein the camera (170), the light source (145), and the processor (180) form part of the electronic device (801), the light source (145) forming a flash for the camera (170), the case (805) having an aperture (806) that houses the deformable membrane (121) and aligns the deformable membrane (121) with the light source (145).

18. The system of claim 17, wherein the portable electronic device (801) comprises a mobile telephone.

19. A method of reconstructing a three-dimensional image comprising:
illuminating a reflective surface (121A) of a deformed membrane (121) using a single-light source (145), wherein the reflective surface (121A) is contoured to a shape of at least a portion of an object (110); and
reconstructing a three-dimensional image of the shape from illumination reflected from the reflective surface (121A) using grayscale mapping.

20. The method of claim 19, wherein the deformable membrane (121) is attached to a flexible material (705).

21. The method of claim 19, wherein the flexible material (705) comprises an isotropically dyed elastomer.

22. The method of claim 19, wherein the deformable membrane (121) encloses a flexible material (705).

23. The method of claim 22, wherein the flexible material comprises a liquid.

24. A system for reconstructing a three-dimensional image comprising:
a deformable membrane (120) that contours to a shape of at least a portion of an object (110), the deformable membrane having a reflective surface (120A);
a camera (170) positioned to receive illumination reflected from the reflective surface (120A);
a light source (141A-H) for illuminating the reflective surface (120A) to produce reflected light onto the camera (170); and
a processor (180) for reconstructing a three-dimensional image of the shape from a video stream corresponding to illumination reflected from the reflective surface (120A).

25. The system of claim 24, wherein the reconstructing a three-dimensional image comprises:
performing a baseline removal on the video stream; and
performing a Karhunen-Loeve Transform after performing the baseline removal.

26. The system of claim 25, wherein reconstructing the three-dimensional image further comprises performing a Fast-Fourier Transform after performing the Karhunen-Loeve Transform.

27. The system of claim 26, wherein reconstructing the three-dimensional image further comprises:
segmenting an output of the Fast Fourier Transform to produce a segmented output; and
fitting the segmented output to three-dimensional image models.

28. The system of claim 27, wherein the image models comprise Gaussian Mixed Models.

29. The system of claim 27, wherein fitting the segmented output is based on Nelder-Mead iterative method.

30. The system of claim 24, wherein reconstructing the three-dimensional image further comprises subtracting unwanted artifacts from images in the video stream.

31. The system of claim 24, wherein the deformable membrane (120) comprises an elastomer or a dyed liquid.

32. A method of reconstructing a three-dimensional image comprising:
illuminating a reflective surface (120A) of a deformed membrane (120) that contours to a shape of at least a portion of an object (110);
receiving illumination reflected from the reflective surface; and
reconstructing a three-dimensional image of the shape of the at least a portion of an object (110) from a video stream corresponding to illumination reflected from the reflective surface (120A).

33. The method of claim 32, wherein the reconstructing a three-dimensional image comprises:
performing a baseline removal on the video stream; and
performing a Karhunen-Loeve Transform after performing the baseline removal.

34. The method of claim 33, wherein reconstructing the three-dimensional image further comprises performing a Fast-Fourier Transform after performing the Karhunen-Loeve Transform.

35. The method of claim 32, wherein reconstructing the three-dimensional image further comprises:
segmenting an output of the Fast Fourier Transform to produce a segmented output; and
fitting the segmented output to three-dimensional image models.

36. The method of claim 35, wherein the image models comprise Gaussian Mixed Models.

37. The method of claim 35, wherein the fitting is based on Nelder-Mead iterative method.

38. The method of claim 32, further comprising subtracting unwanted artifacts from images in the video stream.

39. The method of claim 32, wherein the deformable membrane (120) comprises an elastomer or a dyed liquid.
